(19) United States
(12) Patent Application Publication
(10) Pub. No.: US 2021/0030725 A1
(43) Pub. Date: Feb. 4, 2021

(54) RESTORATION OF TRANSMEMBRANE COPPER TRANSPORT

(71) Applicant: The Board of Trustees of the University of Illinois, Urbana, IL (US)

(72) Inventors: Martin D. Burke, Champaign, IL (US); Chritopher Nardone, Urbana, IL (US); Anddrew Blake, Urbana, IL (US); Anna SantaMaria, Rockville, MD (US); Anthony S. Grillo, Champaign, IL (US); Alexander G. Cioffi, Urbana, IL (US); Yinghuan Lyu, Champaign, IL (US)

(21) Appl. No.: 17/042,544

(22) PCT Filed: Mar. 27, 2019
(86) PCT No.: PCT/US19/24340

§ 371 Date: Sep. 28, 2020

Related U.S. Application Data
(60) Provisional application No. 62/648,662, filed on Mar. 27, 2018.

(51) Int. Cl. A61K 31/435; A61K 31/122; A61K 9/00; A61K 31/423; A61K 31/351; A61P 3/12

(52) U.S. Cl. CPC A61K 31/435; A61K 31/122; A61K 9/0053; A61P 3/12; A61K 31/423; A61K 31/351; A61K 9/0019

(57) ABSTRACT

Disclosed are methods of treating a disease or condition characterized by a deficiency of or a defect in a copper transporter using a small molecule. For example, the method may increase copper transport, or it may increase copper release. Additionally, the small molecule may be hinokitiol, or it may be selected from the group consisting of amphotericin B, calcimycin, nonactin, deferiprone, purpurogallin, and maltol. Also provided is a method of identifying a small molecule capable of treating a disease or condition characterized by a deficiency of or a defect in a copper transporter.

### RELATED APPLICATIONS

This application claims the benefit of priority to U.S. Provisional Application No. 62/648,662, filed Mar. 27, 2018, the contents of which are incorporated herein by reference in their entirety.

### GOVERNMENT SUPPORT

This invention was made with government support under Grant Number GM118185 awarded by the National Institutes of Health. The government has certain rights in the invention.

### BACKGROUND OF THE INVENTION

Copper is one of the least abundant metals in the human body, yet is of great importance in many biological processes, acting as a cofactor for transcriptional regulators, chaperones, cell surface transporters, oxidoreductases, electron transfer, and free radical scavengers. Copper homeostasis is maintained by passive and active transport proteins that shuttle copper into, within, and/or out of cells while maintaining the labile pool at exquisitely low levels to minimize toxicity of this highly redox active metal. Two rare genetic disorders, Menkes syndrome and Wilson's disease, manifest due to mutations in the copper transporting P-type ATPases, ATP7A and ATP7B, respectively. In Menkes syndrome, loss of ATP7A, which is normally localized to the basolateral membrane of enterocytes, leads to reduced dietary copper absorption into the bloodstream and hyperaccumulation of copper in the gut. Most patients with Menkes syndrome die by the third year of life, with infants exhibiting neurological, developmental, vascular, skeletal, and pleiotropic abnormalities shortly after birth. In Wilson's disease, loss of ATP7B, which is expressed as a transmembrane protein in hepatocytes, leads to reduced copper excretion and thus hyperaccumulation of copper in the liver. ATP7B has also recently been implicated in intestinal copper homeostasis through copper sequestration and cytosolic buffering in intracellular copper-containing vesicles. Similar to Menkes syndrome, patients with Wilson's disease exhibit pleiotropic abnormalities due to systemic copper overload, with major complications including liver failure, as well as neurological and psychiatric manifestations, such as akinetic-rigid syndrome similar to Parkinson's disease, tremor, ataxia, chorea, micrographia, behavioral changes, and more.

Treatment options for these currently incurable diseases fail to address the underlying molecular defect: the loss of transmembrane copper transport. Given that oral administration is refractory, treatments for Menkes syndrome are primarily focused on copper supplementation parenterally or subcutaneously, coupled with a heavy emphasis on symptom management. As for Wilson's disease, current therapies focus on removal of excess copper by water-soluble chelating agents that are then excreted in the urine. Two clinically approved copper chelators, penicillamine (Cuprimine®) and triethylenetetramine (Syprine®), have formed the basis for much of Wilson's disease treatment over the past 50 years. Other options include zinc supplementation to reduce intestinal copper absorption, more potent chelators such as tetrathiomolybdate salts (Decuprate®), and second line drugs including 2,3-dimercapto-1-propanol. However, these approaches all fail to directly address the issue of a missing transport protein, and trigger a number of side effects including neurological deterioration.

A lipophilic small molecule natural product, called hinokitiol, has the remarkable capacity to autonomously transport iron across lipid bilayers and replace the missing function of key iron transporting proteins. Hinokitiol was able to restore yeast cell physiology to a strain missing a crucial iron uptake complex. Hinokitiol treatment was also able to restore gut iron absorption and iron recycling to promote hemoglobinization in mammalian cells, zebrafish, rats, and multiple mouse models deficient in a number of iron transporting proteins. Importantly, hinokitiol not only restored iron flux, but maintained the bioavailability of the iron for intracellular processes.

Normally, the cytosolic labile copper pool is roughly 10 orders of magnitude smaller than the labile iron pool. Thus, it is not apparent that hinokitiol should be able to selectively mobilize copper. In a diseased state, a build-up of copper occurs in the gut or liver to greater than 5 times that of normal physiology, thereby setting the stage for site- and direction-selective transmembrane copper transport. However, iron is still many orders of magnitude greater in concentration, suggesting that hinokitiol might not be effective for copper transport.

### SUMMARY OF THE INVENTION

Provided is a molecular prosthetic to address the copper imbalance issue that occurs in Menkes and Wilson's diseases, and occipital horn syndrome. Specifically, small molecules, such as hinokitiol, can serve as a functional surrogate for missing protein copper transporters and thereby restore physiology in Ctr1Ctr3 deficient yeast. It is shown that hinokitiol is exceptionally capable of forming lipid-soluble complexes with copper. It may also possess exceptional capacity to restore copper homeostasis in the nervous system via mobilizing copper from neurons and/or other cells in the CNS, as the brain is normally more difficult to access by water-soluble compounds. These studies support the untapped potential of treating disorders related to transmembrane copper transport with small molecules, such as hinokitiol.

### BRIEF DESCRIPTION OF THE DRAWINGS

FIG. 1 illustrates the mechanistic aspects of Menkes disease and Wilson's disease, diseases of copper transport in which ATP7A/7B is missing.

FIG. 2 shows that hinokitiol binds to and sequesters copper with a greater affinity than for Fe, Mn, Co, Ni, or Zn.

FIG. 3 shows that hinokitiol transports copper across membranes faster than it transports Fe, Mn, Co, Ni, or Zn.

FIG. 4A compares hinokitiol to a number of copper chelators, including those currently used in the clinic for Wilson's disease: penicillamine, trientine, and tetrathiomolybdate salts, along with dimercaptopropanol and dimercaptosuccinic acid.

FIG. 4B is a 3-D representation of the chelation between copper and hinokitiol.

FIG. 4C shows hinokitiol, in contrast to water-soluble copper chelators, autonomously promotes the release of copper from a model POPC liposome.

FIG. 5A is a schematic illustration of copper transporter-deficient yeast that are unable to grow, which are then able to grow after addition of a small molecule that promotes the transmembrane copper transport process; the small molecule replaces the function of the copper transporter CTR1/CTR3 in DELctr1/DELctr3 deficient yeast.

FIG. 5B is a graph showing hinokitiol restored growth of Dctr1Dctr3 yeast in a copper concentration-dependent manner.

FIG. 5C is a graph showing hinokitiol, which restored growth in copper transporter deficient Dctr1Dctr3 yeast, does not restore growth in iron transporter-deficient yeast under the same media conditions.

FIG. 6 illustrates vigorous rescue by hinokitiol of a strain of yeast missing the intracellular ATP-driven copper pump CCC2 (DELccc2 yeast strain) on solid media.

FIG. 7 are two graphs showing vigorous rescue by hinokitiol of a strain of yeast missing the intracellular ATP-driven copper pump CCC2 (DELccc2 yeast strain) in liquid culture.

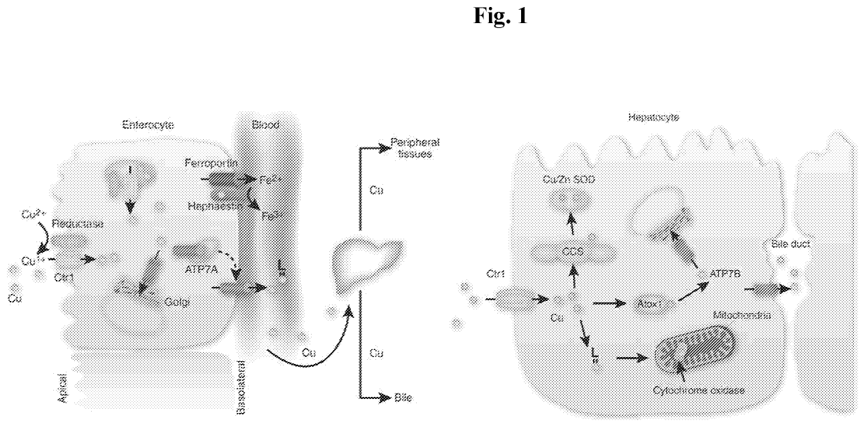

### DETAILED DESCRIPTION OF THE INVENTION

The invention stems from the findings that small molecules, e.g., hinokitiol, can serve as functional surrogates for missing protein copper transporters and thereby restore physiology in yeast deficient in copper transporting complex Ctr1Ctr3. Hence, the invention relates to the untapped potential of treating disorders related to transmembrane copper transport with small molecules, such as hinokitiol.

As used herein, the term “treat” or “treatment” is defined as the application or administration of a compound, alone or in combination with a second compound, to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, in order to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder). In the case of wound healing, a therapeutically effective amount is an amount that promotes healing of a wound.

As used herein, an amount of a compound effective to treat a disorder, or a “therapeutically effective amount” refers to an amount of the compound which is effective, upon single or multiple dose administration to a subject or a cell, in curing, alleviating, relieving or improving one or more symptoms of a disorder. In the case of wound healing, a therapeutically effective amount is an amount that promotes healing of a wound.

Administration “in combination with” one or more further therapeutic agents includes simultaneous (concurrent) and consecutive administration in any order.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, the mode of administration, the bioavailability of the particular compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

“Modulating” or “modulate” refers to the treating, prevention, suppression, enhancement or induction of a function, condition or disorder.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used in the present disclosure and claims, the singular forms “a,” “an,” and “the” include plural forms unless the context clearly dictates otherwise.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described herein with the language “comprising,” otherwise analogous embodiments described in terms of “consisting of” and/or “consisting essentially of” are also provided.

As used herein, “subject” refers to a warm blooded animal such as a mammal, such as a human, or a human child, which is afflicted with, or has the potential to be afflicted with one or more diseases and disorders described herein.

For example, provided herein are methods of treating various disease or condition characterized by a deficiency of or a defect in a copper transporter in mammals (including humans and non-humans), comprising administering to a patient in need thereof a compound of the invention, or a pharmaceutically acceptable salt thereof. Such various disease or condition characterized by a deficiency of or a defect in a copper transporter include: Menkes syndrome, Wilson's disease, or occipital horn syndrome. Copper transporting proteins that are deficient in Menkes and Wilson's diseases are active ATP-driven pumps, so it is remarkable that a passive small molecule can substitute for an active ATP-driven protein pump.

“Hinokitiol”, as used herein, is represented by the structural formula:

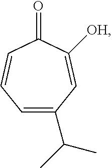

and is also referred to as “β-thujaplicin”, “2-hydroxy-6-propan-2-ylcyclohepta-2,4,6-trien-1-one”, and “4-Isopropyltropolon”.

One aspect of the invention relates to a method of treating a disease or condition characterized by a deficiency of or a defect in a copper transporter, comprising administering to a subject in need thereof a therapeutically effective amount of a small molecule, thereby treating the disease or condition characterized by a deficiency of or defect in an copper transporter.

In certain embodiments, the small molecule is selected from the group consisting of amphotericin B (AmB), calcimycin, nonactin, deferiprone, purpurogallin, and maltol, and any combination thereof.

In certain embodiments, the small molecule is selected from the group consisting of calcimycin, deferiprone, purpurogallin, and maltol, and any combination thereof.

In certain embodiments, the small molecule is hinokitiol.

In some embodiments, the small molecule is administered as a pharmaceutical composition.

The pharmaceutical compositions of the present invention can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

In certain embodiments, the small molecule is administered systemically. In certain embodiments, the small molecule is administered orally. In certain embodiments, the small molecule is administered intravenously.

Compounds of the invention can be combined with other therapeutic agents. The compound of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compound of the invention, when the administration of the other therapeutic agents and the compound of the invention is temporally separated. The separation in time between the administration of these compounds may be a matter of minutes or it may be longer.

Examples of other therapeutic agents that may be administered with the compounds of the invention include water-soluble copper chelators, such as D-penicillamine, triethylenetetramine, meso-2,3-dimercaptosuccinic acid, 2,3-dimercaptopropanol, and ammonium tetrathiomolybdate. Additionally, copper supplements may be co-administered with a compound of the invention. In certain embodiments, the invention relates to co-administration of a compound of the invention and copper chelator therapy.

Thus, another aspect of the invention provides a pharmaceutical composition comprising a therapeutically effective amount of a compound of the invention; and a second therapeutic agent selected from the group consisting of D-penicillamine, triethylenetetramine, meso-2,3-dimercaptosuccinic acid, 2,3-dimercaptopropanol, and ammonium tetrathiomolybdate.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be, for human subjects, from about 0.0001 milligrams/kg per day, 0.001 milligrams/kg per day, or 0.01 milligrams/kg per day to about 100 milligrams/kg per day or 1000 milligrams/kg per day. It is expected that oral doses in the range of 0.5 to 100 milligrams/kg, in one or several administrations per day, will yield the desired results. Dosage may be adjusted appropriately to achieve desired drug levels sufficient to achieve or maintain a desired therapeutic effect, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds. The compounds may be administered once per week, several times per week (e.g., every other day), once per day or multiple times per day, depending upon, among other things, the mode of administration, the specific indication being treated and the judgment of the prescribing physician. In one embodiment, a compound of the invention may typically be administered at a dose from 0.1 mg/kg/day to 125 mg/kg/day. For example, a compound of the invention may be administered at a dosage of about 25 mg/kg/day, 50 mg/kg/day, 75 mg/kg/day, or 100 mg/kg/day.

Determination of an effective dosage of a compound for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

Pharmaceutical compositions comprising the compound of the invention may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

For use in therapy, an effective amount of the compound of the invention can be administered to a subject by any mode that delivers the compound of the invention to the desired surface. Administering the pharmaceutical composition of the present invention may be accomplished by any means known to the skilled artisan. Routes of administration include but are not limited to oral, buccal, nasal, rectal, vaginal, ocular, topical, intravenous, intramuscular, intraperitoneal, subcutaneous, transdermal, intrathecal, direct injection (for example, into an abscess), mucosal, inhalation, and insufflation.

For oral administration, the compounds (i.e., compounds of the invention, and other therapeutic agents) can be formulated readily by combining the active compound(s) with pharmaceutically acceptable carriers well known in the art. Such carriers enable the compounds of the invention to be formulated as tablets, pills, dragees, lozenges, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, binding agents, fillers, lubricants, disintegrants, and wetting agents. Suitable fillers include sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

In addition to the formulations described above, for prolonged delivery, the compounds may also be formulated as a depot preparation for administration by, for example, implantation or intramuscular injection. Such long acting formulations may be formulated with suitable polymeric or hydrophobic materials (for example, as an emulsion in an acceptable oil) or ion exchange resins, or as sparingly soluble derivatives, for example, as a sparingly soluble salt. Alternatively, transdermal delivery systems manufactured as an adhesive disc or patch which slowly releases the compound for percutaneous absorption may be used. To this end, permeation enhancers may be used to facilitate transdermal penetration of the compound.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group. Typically, such salts are more soluble in aqueous solutions than the corresponding free acids and bases, but salts having lower solubility than the corresponding free acids and bases may also be formed.

The compounds may alternatively be formulated in the pharmaceutical composition per se, or in the form of a hydrate, solvate, or N-oxide.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

Methods and Uses

As shown herein, the small molecules administered to a patient in need thereof are useful in restoring physiology, as well as restoring the transport of copper across monolayers in subject deficient in copper transporter.

Accordingly, the invention provides methods for the treatment or prevention of a disease, disorder, or condition associated with deficiency of or a defect in a copper transporter, comprising administering to a subject in need thereof a therapeutically effective amount of at least one small molecule, or a pharmaceutical composition thereof.

In certain embodiments, the disease, disorder, or condition associated with a deficiency of or a defect in a copper transporter is Menkes syndrome, Wilson's disease, or occipital horn syndrome.

In some aspects, the invention provides methods for increasing copper transport in a subject in need thereof, comprising administering to the subject a therapeutically effective amount of a small molecule, or a pharmaceutical composition thereof.

In some aspects, the invention of provides methods of increasing growth, comprising administering to the subject in need thereof a therapeutically effective amount of a small molecule, or a pharmaceutical composition thereof.

In some aspects, the invention of provides methods of increasing physiology in a subject in need thereof, comprising administering to the subject a therapeutically effective amount of a small molecule, or a pharmaceutical composition thereof.

In some aspects, the invention provides methods of increasing copper release or absorption in a patient in need thereof, comprising administering to the subject a therapeutically effective amount of a small molecule.

In some aspects, the invention provides methods of increasing in vitro one or more of copper transport or physiology in a cell.

In some aspects, the invention provides methods of increasing ex vivo one or more of copper transport or physiology in a cell or organ.

In certain embodiments, the small molecule is selected from the group consisting of amphotericin B (AmB), calcimycin, nonactin, deferiprone, purpurogallin, and maltol, and any combination thereof.

In certain embodiments, the small molecule is selected from the group consisting of calcimycin, deferiprone, purpurogallin, and maltol, and any combination thereof.

In certain embodiments, the small molecule is hinokitiol.

Another aspect of the invention relates to methods of screening for a small molecule capable of treating a disease or condition characterized by a deficiency of or a defect in a copper transporter in a subject in need thereof, comprising the steps of: determining an increase in copper binding and transport; determining an increase in restoration of growth; and determining an increase in physiology in human disease-relevant system.

In certain embodiments, a small molecule identified by such a method is efficacious in treating a disease or condition characterized by a deficiency of or a defect in a copper transporter.

Alternatively, the efficacy of a small molecule for treating a disease or condition characterized by a deficiency of or a defect in a copper transporter can be determined via measuring copper binding transport, measuring restoration of growth, and measuring physiology in human disease-relevant system. In certain such embodiments, the binding transport, restoration of growth, and physiology in human disease-relevant system can be measured prior to and after administration of a small molecule. When the binding transport, restoration of growth, and physiology in human disease-relevant system increase after administration of a small molecule, such an agent is efficacious in treating a disease or condition characterized by a deficiency of or a defect in a copper transporter.

In some embodiments, the subject receiving treatment is a mammal. For instance, the methods and uses described herein are suitable for medical use in humans. Alternatively, the methods and uses are also suitable in a veterinary context, wherein the subject includes but is not limited to a rat, dog, cat, horse, cow, sheep and goat.

In some embodiments, the subject is deficient in a copper transporter. In some embodiments, the subject is deficient in copper transporting P-type ATPases (ATP7A or ATP7B).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Exemplary Materials & Methods

### Hinokitiol-Promoted Release of Different Divalent Metal

Hinokitiol-promoted release of different divalent metals from POPC liposomes was performed by tracking the quenching of PhenGreen (Fisher P14312) similar to previously reported. Liposomes were prepared as described above with an internal buffer consisting of either 10 mM ascorbate in a 5 mM MES/Tris buffer at pH=7.0 (for Fe²⁺), 10 mM citrate in a 5 mM MES/Tris buffer at pH=7.0 (for Cu²⁺), or a 5 mM MES/Tris buffer at pH=7.0 (for Mn²⁺, Co²⁺, and Zn²⁺). In all cases, liposomes were prepared using 5 mM of either FeCl₂, MnCl₂, CoCl₂, NiCl₂, ZnCl₂, or CuCl₂ added to the internal buffer. The external buffer was a 5 mM MES/Tris buffer at pH=7.0 containing 10 μM PhenGreen (from 1000× stock in DMSO). The liposome suspension was diluted to 1 mM of phosphorus. The liposome suspension was transferred to a 96-well plate, and either DMSO or 2 μM hinokitiol (from a 40× stock in DMSO) was added at t=2 min. The fluorescence was monitored with excitation at 500 nm and emission at 530 nm over 1 hour. After one hour, the liposomes were lysed with Triton-X and the fluorescence was recorded. In all cases, quenching of fluorescence was observed in the DMSO-treated liposomes after lysis, which reached similar levels to that for hinokitiol-treated liposomes before lysis (except for Mn²⁺ where no efflux was observed in Hino-treated liposomes; fluorescence quenching was observed after lysis for Mn²⁺). The DMSO-treated and hinokitiol-treated liposomes had similar fluorescence quenching levels after lysis. The total amount of metal efflux was determined using standard curves of fluorescence quenching in external buffer with 10 μM PhenGreen and known concentrations of each metal. The t_{1/2} values were calculated using an asymptotic fit in OriginPro. The t_{1/2} values indicate the time required to reach half of the maximum metal efflux for each metal.

Growth Rescue of Δctr1Δctr3 Yeast Strain with Small Molecules.

Growth rescue in yeast was performed using 10 μM hinokitiol in SD media in a 96-well plate unless otherwise noted. Wild type and DELctr1DELctr3 yeast strain controls treated with vehicle (DMSO) or hinokitiol were performed under identical conditions using a normal iron-containing, no-copper containing SD media, supplemented with 1.0 μM CuCl₂. Yeast were grown overnight in YPD media and diluted at an OD600 of 0.1 in SD media, diluted 10-fold, and incubated at 30° C. with continuous shaking (200 rpm). The OD600 was obtained 48 hours after inoculation unless otherwise noted. Small molecule dose-response with hinokitiol was determined by addition of the small molecule (40× stock solution in DMSO) to give the indicated final concentrations. Copper dose-response studies were performed in low copper SD media without CuCl₂ containing the indicated concentrations of hinokitiol (from a 40× stock solution in DMSO). For dose-dependent hinokitiol-promoted rescue at increasing dosages of CuCl₂, SD media was made containing the indicated concentrations of CuCl₂ from 1000× CuCl₂ stocks before adding hinokitiol (40× stock solution in DMSO) to give the indicated final concentrations.

Growth Rescue of DELccc2 Yeast Strain with Small Molecules in Solid and Liquid Media

Growth rescue in yeast was performed on SD-agar plates in 50 mM MES/Tris buffer at pH=7.0 containing 2% agarose gel, and 10 μM hinokitiol (from 40× stock in DMSO). Wild type and DELccc2 yeast strain controls treated with vehicle (DMSO) were performed under identical conditions using a no-copper containing SD media supplemented with 0.5 μM CuCl₂ in the absence of hinokitiol. Yeast were grown overnight in YPD media and diluted to an optical density at 600 nM (OD₆₀₀) of 1.0 in low copper SD media before 10-fold serial dilution and inoculation of these yeast suspensions (10 μL per dot) onto the low copper SD-agar plates described above containing either DMSO vehicle or hinokitiol (10 μM from 40× DMSO stock). For disc diffusion assays, yeast were grown overnight in YPD media and diluted to an OD₆₀₀=0.1 in low copper SD media and streaked onto low copper SD-agar plates containing 0.5 μM CuCl₂. Disc diffusion assays were performed using either DMSO or 10 mM stock solutions (in DMSO) of hinokitiol on low copper SD-agar plates containing 0.5 μM CuCl₂ streaked with the appropriate yeast strain (from OD600=0.1 in low copper SD media). Images were taken 48 hours after inoculation and incubation at 30° C. unless otherwise noted.

Growth rescue in yeast was performed using 10 μM hinokitiol in SD media in a 96-well plate unless otherwise noted. Wild type and DELccc2 yeast strain treated with vehicle (DMSO) or hinokitiol were performed under identical conditions using a normal iron-containing, no-copper containing SD media, this time supplemented with 1.5 or 2 μM CuCl₂. Yeast were grown overnight in YPD media and diluted at an OD600 of 0.1 in SD media, diluted 10-fold, and incubated at 30° C. with continuous shaking (200 rpm). The OD600 was obtained 48 hours after inoculation unless otherwise noted. Small molecule dose-response with hinokitiol was determined by addition of the small molecule (40× stock solution in DMSO) to give the indicated final concentrations. Copper dose-response studies were performed in low copper SD media without CuCl₂ containing the indicated concentrations of hinokitiol (from a 40× stock solution in DMSO). For dose-dependent hinokitiol-promoted rescue at increasing dosages of CuCl₂, SD media was made containing the indicated concentrations of CuCl₂ from 1000× CuCl₂ stocks before adding hinokitiol (40× stock solution in DMSO) to give the indicated final concentrations.

### Example 1: Hinokitiol Binds to and Sequesters Copper with a Great Affinity and Rapidly Transports Copper Across Membranes

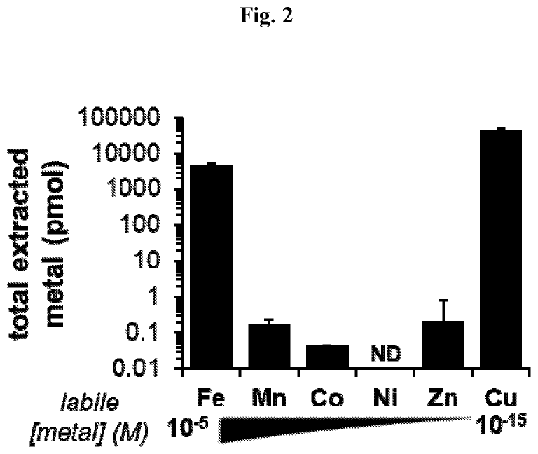

Hinokitiol binds to and sequesters copper with a greater affinity than any other metal (including iron). Stoichiometric competition experiments with 1 mM of each divalent metal and 1 mM hinokitiol in a 10 mM Mes/Tris buffer in 1:1 MeOH:H2O at pH=7.0. Hinokitiol binds many metals by ICP-MS quantification of extracted hinokitiol-bound metal. The metal in the organic layer was determined to be in the order of Cu>Fe>Mn≈Zn>Co>Ni. ND=Not detected. N=6. (FIG. 2)

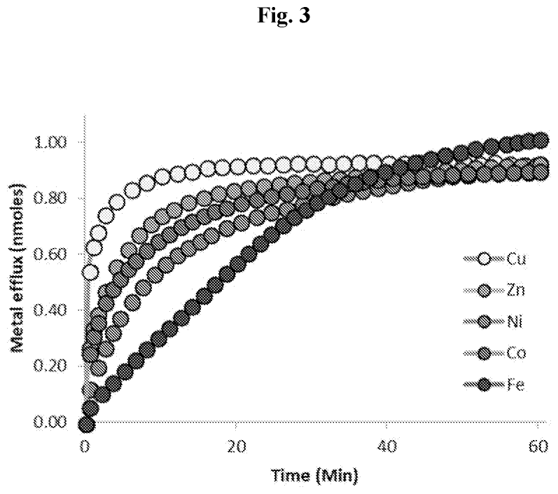

Hinokitiol transports copper across membranes faster than any other metal. Hinokitiol (10 μM) rapidly promotes the efflux of multiple divalent metals from POPC liposomes as determined by a PhenGreen assay. In these studies, hinokitiol was unable to transport Mn_{II}to any observable degree. Quantification of metal release was done by comparison of fluorescence quenching to a standard curve for each metal. The reciprocal half-lives for efflux were determined to be in the order of Cu>Zn>Co>Ni>Fe>>Mn. N=3. (FIG. 3)

### Example 2: The Capacity of Hinokitiol to Bind and Transport Copper

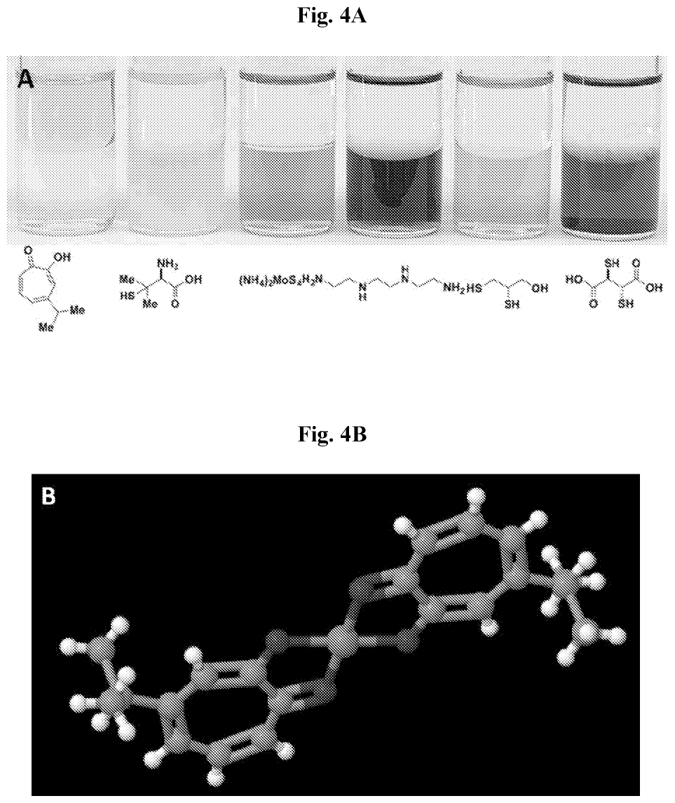

Biophysical experiments were performed to better understand the capacity of hinokitiol to bind and transport copper. This small molecule readily binds copper to form a hinokitiol:copper complex, as evidenced by a shift in the ultraviolet-visible (UV-vis) spectra upon titrating with copper (II) chloride (FIG. 4B). Unlike the aforementioned water-soluble copper chelators, the hinokitiol:copper complex primarily partitioned into the nonpolar solvents over water (FIG. 4A). To expand, 95% of the hinokitiol:copper complex partitioned into octanol over water as opposed to >85% of the currently available water-soluble chelators partitioned into water over octanol. Hinokitiol also transported copper (II) across liposomal membranes, whereas the suite of chelating agents showed minimal transport relative to controls (FIG. 4C).

### Example 3: Growth Recue of Δctr1Δctr3 Yeast Missing the Passive Copper Transporters

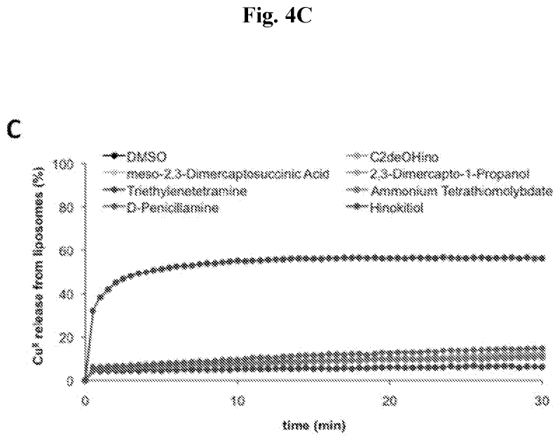

Given these highly encouraging results, the capacity for hinokitiol to restore transmembrane copper transport and therefore growth in a train of Saccharomyces cerevisiae, Δctr1Δctr3, missing the passive copper transporters Ctr1Ctr3 (FIG. 5A) was tested. Under respiratory and non-permissive conditions, hinokitiol dose-dependently restored yeast growth up to wild type control levels with maximum restoration occurring at intermediate concentrations of hinokitiol (FIG. 5B). Additionally, growth restoration was copper dependent and aligned with the extracellular copper gradient established in the media (FIG. 5B). Unlike in the case of Δctr1Δctr3, hinokitiol was unable to restore growth to iron transporter-deficient yeast missing the FetFtr1 transport complex under respiratory and non-permissive copper conditions (FIG. 5C). These results are consistent with hinokitiol-mediated transport of copper, rather than iron, across yeast membranes leading to restoration of cell growth. These results support the potential of hinokitiol to transport copper and restore homeostasis in people with Menkes and Wilson's disease.

### Example 4: Growth Recue of DELccc2 Yeast Strain Missing the Intracellular ATP-Driven Copper Pump CCC2

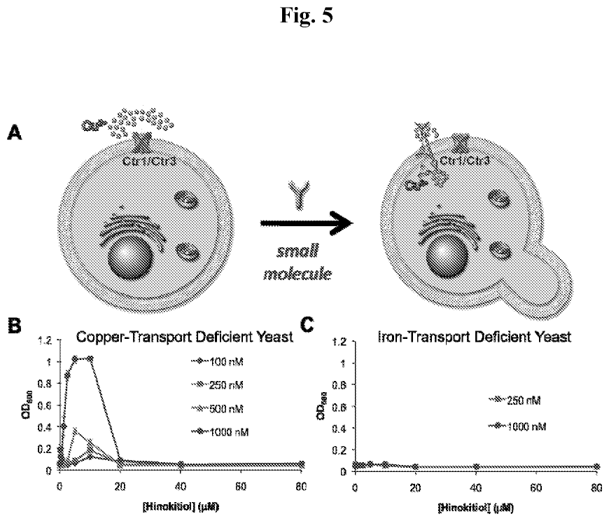

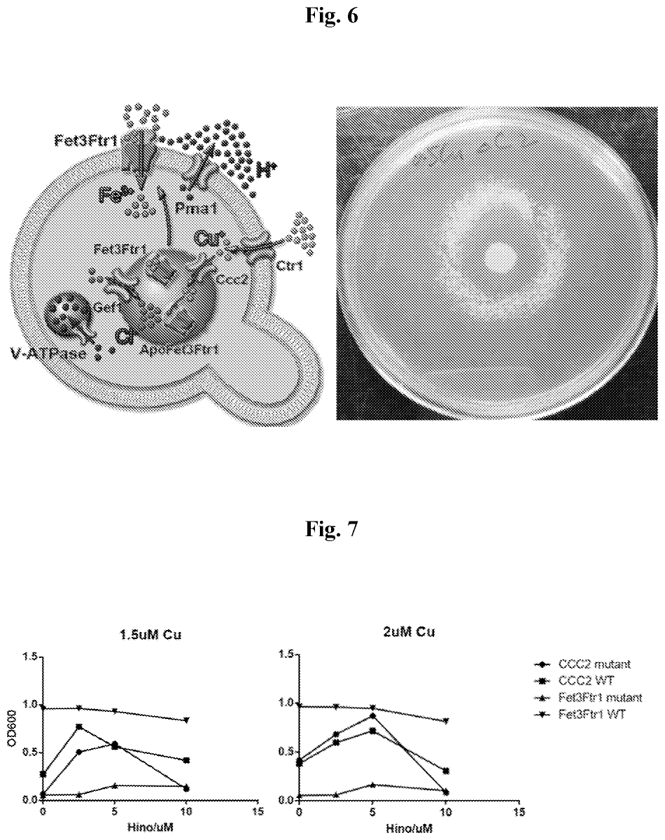

Growth rescue experiment of a strain of yeast missing the intracellular ATP-driven copper pump ccc2 (DELccc2 yeast strain) was designed and performed in order to test if hinokitiol can replace an active (ATP-driven) copper transporter. A direct consequence of this missing transporter is an inability to load copper into the fet3/ftr1 iron transport complex, stunting yeast growth. Vigorous rescue of the ccc2 yeast both on solid media (FIG. 6) and in liquid culture (FIG. 7)—in conditions where fet3/ftr1 yeast rescue was not afforded. This shows that hinokitiol can replace a missing ATP-driven active copper transporter. This provides direct evidence for the capability of hinokitiol to serve as a replacement for the defective ATP7A & ATP7B transporters in Menkes & Wilson's disease, and the first evidence that a passively diffusing small molecule can replace an active transport protein.

### INCORPORATION BY REFERENCE

All U.S. patents and published U.S. and PCT patent applications cited herein are hereby incorporated by reference in their entirety as if each patent or patent application publication was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating a disease or condition characterized by a deficiency of or a defect in a copper transporter, comprising administering to a subject in need thereof a therapeutically effective amount of a small molecule, thereby treating the disease or condition.

2. The method of claim 1, wherein the disease or condition characterized by a deficiency of or defect in a copper transporter is Menkes syndrome, Wilson's disease, or occipital horn syndrome, or any combination thereof.

3. A method of increasing copper transport, comprising administering to a subject in need thereof an effective amount of a small molecule.

4. A method of increasing physiology, comprising administering to a subject in need thereof an effective amount of a small molecule.

5. A method of increasing copper release or absorption, comprising administering to a subject in need thereof an effective amount of a small molecule.

6. The method of any one of the claims 1-5, wherein the small molecule is selected from the group consisting of amphotericin B (AmB), calcimycin, nonactin, deferiprone, purpurogallin, and maltol, and any combination thereof.

7. The method of any one of claims 1-5, wherein the small molecule is selected from the group consisting of calcimycin, deferiprone, purpurogallin, and maltol, and any combination thereof.

8. The method of any one of claims 1-5, wherein the small molecule is hinokitiol.

9. The method of any one of claims 1-8, wherein the small molecule is administered systemically.

10. The method of any one of claims 1-8, wherein the small molecule is administered orally.

11. The method of any one of claims 1-8, wherein the small molecule is administered intravenously.

12. The method of any one of claims 1-11, wherein the subject is a mammal.

13. The method of any one of claims 1-11, wherein the subject is a human.

14. The method of any one of claims 1-13, wherein the subject is deficient in copper transporting P-type ATPase ATP7A or ATP7B, or a combination of both.

15. A method of increasing copper transport or physiology in a cell in vitro, comprising contacting the cell with an effective amount of a small molecule.

16. A method of increasing copper transport or physiology in an organ ex vivo, comprising contacting the organ with an effective amount of a small molecule.

17. The method of claim 15 or 16, wherein the small molecule is selected from the group consisting of amphotericin B (AmB), calcimycin, nonactin, deferiprone, purpurogallin, and maltol, and any combination thereof.

18. The method of any one of claim 15 or 16, wherein the small molecule is selected from the group consisting of calcimycin, deferiprone, purpurogallin, and maltol, and any combination thereof.

19. The method of claim 15 or 16, wherein the small molecule is hinokitiol.

20. A method of identifying a small molecule capable of mitigating a cellular deficiency of or a cellular defect in a copper transporter, comprising the steps of:
a) contacting a cell with a small molecule; and
b) determining an increase in copper binding and transport in the cell;
wherein the cell has a cellular deficiency of or a cellular defect in a copper transporter.

21. The method of claim 20, further comprising determining an increase in or restoration of cell growth.

22. The method of claim 20 or 21, further comprising determining an improvement to or increase in physiology in the cell.
